(19) United States
(12) Patent Application Publication
(10) Pub. No.: US 2025/0009212 A1
(43) Pub. Date: Jan. 9, 2025

(54) Endoscope

(71) Applicant: Heck & Reddy Innovations, LLC, Georgetown, TX (US)

(72) Inventors: Benjamin Heck, Georgetown, TX (US); Chakravarthy Reddy, Salt Lake City, UT (US)

(21) Appl. No.: 18/764,029

(22) Filed: Jul. 3, 2024

Related U.S. Application Data
(60) Provisional application No. 63/511,812, filed on Jul. 3, 2023.
(60) Provisional application No. 63/542,092, filed on Oct. 3, 2023.

(51) Int. Cl. A61B 1/015; A61B 1/00; A61B 1/005; A61B 1/267

(52) U.S. Cl. CPC A61B 1/015; A61B 1/00042; A61B 1/00068; A61B 1/00108; A61B 1/00128; A61B 1/0051; A61B 1/2676

(57) ABSTRACT

A portable endoscope with controlled suction including a proximal component and a distal component. The proximal component includes a handle and the distal component includes a flexible insertion tube. The insertion tube includes an internal working channel extending from the distal end to the proximal component. A powered suction system includes a fluid circuit in fluid communication with the internal working channel. The powered suction system provides suction to the distal end and includes a source of negative pressure from a pump and a liquid trap in the fluid circuit configured to separate liquids from the air delivered to the source of negative pressure. The endoscope includes a suction control system configured to use a sensed pressure to regulate the negative pressure applied to the working channel.

### CROSS-REFERENCE TO RELATED APPLICATIONS

This application claims the benefit of U.S. Provisional Patent Applications Nos. 63/511,812 filed 3 Jul. 2023, titled Suction Inducing Endoscope and 63/542,092 filed 3 Oct. 2023, titled Endoscope Accessory. The foregoing applications are incorporated herein by reference in their entirety.

### TECHNICAL FIELD

The present disclosure relates to endoscopes with improved and/or controlled suction and computer implemented methods for controlling the pressure of the endoscopes.

### BACKGROUND

A bronchoscope is a medical instrument which is placed in the lower respiratory tract of a patient through the mouth or the nose. It is used for observing the pathological changes of lung lobes, segments and subsegments of bronchus, biopsy sampling, bacteriological examination and cytological examination, and can be used for photographing, teaching and dynamic recording in cooperation with a display system. Bronchoscopes typically have a flexible insertion tube with an imager and light source at the tip for viewing the airways of the lung while performing procedures in the airway. The flexible tube includes a working channel that can be used to introduce an instrument into the lungs through an accessory port and perform procedures on the lung tissue. These procedures typically require suction. For example, bronchoalveolar lavage is a procedure where fluid is introduced into the lung and then suctioned out.

### BRIEF DESCRIPTION OF THE DRAWINGS

Non-limiting and non-exhaustive implementations of the disclosure are described with reference to the following figures, wherein like reference numerals refer to like parts throughout the various views unless otherwise specified. It will be appreciated by those of ordinary skill in the art that the various drawings are for illustrative purposes only. The nature of the present disclosure, as well as other embodiments in accordance with this disclosure, may be more clearly understood by reference to the following detailed description, to the appended claims, and to the several drawings.

FIG. 1 is a perspective view of bronchoscope according to one embodiment of the invention;

FIG. 2 is another view of the bronchoscope of FIG. 1 with the distal component truncated to better illustrate the proximal component;

FIG. 3 is an exploded view of the bronchoscope of FIG. 2 showing the internal parts of the proximal component;

FIG. 4 illustrates the principle internal components of the proximal component that make up the fluid circuit of the bronchoscope of FIG. 2;

FIG. 5 shows an exploded view of the manifold and circuit board of the endoscope shown in FIG. 4;

FIG. 6 is a cross section of FIG. 2 showing a cavity for positing tubing and wires above the flexion assembly;

FIG. 7 is an exploded view of a portion of the components shown in FIG. 3 showing the flexion components in more detail;

FIGS. 8A-8F illustrate a user input element for controlling pressure according to one embodiment of the invention;

FIGS. 9A-9D illustrate a negative pressure regulator that can be used in an alternative embodiment of an endoscope according to one embodiment of the invention;

FIGS. 10A-10E illustrate the flexible portion, camera, and LEDs of the insertion tube of the endoscope of FIG. 1;

FIG. 11 illustrates an alternative embodiment of an endoscope according to the invention where components of the pressure control system are in an external component of the bronchoscope;

FIG. 12 illustrates an endoscope according to another embodiment of the invention showing a support bracket that can be rotatably attached to a proximal component and provide physical support for the liquid trap 16 to prevent it from undesired movements; and

FIG. 13 is a flow diagram describing aspects of the computer executable instructions for operating an endoscope according to an embodiment of the invention.

### DETAILED DESCRIPTION

Provided herein are endoscopes for performing medical procedures that utilize suction. In several of the embodiments, the endoscopes use a pressure sensor and a pressure control system to regulate a source of negative pressure. The controlled suction gives the clinician control to select negative pressures that are higher and/or lower than the typical wall suction pressure used with traditional endoscopes. The endoscopes include a negative pressure pump that generates a desired negative air pressure for use in suctioning through the working channel of the endoscope. The endoscope may also include a suction control system responsive to user input for selectively lowering or raising the pressure. Examples of such user input include, but is not limited to, a position sensor, voice control, adjustment of mechanical mechanisms, and external buttons.

To control the suction pressure in the working channel, some embodiments of the invention use a liquid trap. The liquid trap is positioned between the working channel and the source of the negative pressure. The fluid trap can facilitate the suctioning in several ways.

First, the trap can prevent liquids from passing through the pump. This allows the use of a pump configured to pump air (i.e., a gas pump). Air is compressible and easier to move. Although not required, the air pressure pump can be incorporated into the handle of the endoscope.

Secondly, the liquid trap can provide a source of air for sampling the air pressure applied to the working channel. Trapping the liquid coming from the patient via the working channel produces a source of air that can be used for sensing pressure. The pressure sensor can be in fluid connection with the air in the trap using a tube or placing the sensor in the trap. Sensing the pressure “applied to” the trap includes sensing pressure using a tube that is sized to minimize pressure loss to the sensor or is calibrated to account for pressure loss between the sensor and the trap.

In one embodiment of the invention, a portable endoscope with controlled suction includes a proximal component and a distal component. The proximal component includes a handle and the distal component includes a flexible insertion tube extending from the proximal component to a distal end. In some embodiments, the insertion tube may be rigid. The insertion tube includes an internal working channel extending from the distal end to the proximal component. A powered suction system includes a fluid circuit in fluid communication with the internal working channel of the proximal component, the powered suction system provides suction to the distal end. The powered suction system includes a source of negative pressure from a pump. The endoscope includes a liquid trap in the fluid circuit. The liquid trap is in fluid communication with the source of negative pressure.

The endoscope can include a pressure sensor configured to sense the negative air pressure applied to the liquid trap. A suction control system including an electrical processing unit electrically coupled to the pressure sensor, the suction control system is configured to use the sensed pressure to regulate the negative pressure applied to the working channel.

In another embodiment, the proximal component of the endoscope includes one or more user input elements that provide input to the suction control system to selectively adjust the regulated negative pressure applied to the working channel of the distal component. The electrical processing unit is responsive to the user input for regulating suction pressure.

The portable endoscope can include a suction control system that regulates the pressure applied to the working channel by modulating the power delivered to the pump, which modulates the speed of the pump. Alternatively, the suction control system regulates the pressure applied to the working channel by modulating the release of pressure in the fluid circuit. For example, the pressure can be released mechanically using a negative pressure regulator, or can be released using an electrical control system that uses a pressure sensor and computer implemented instructions to generate a signal delivered to an electronically controlled valve that is opened and closed to modulate the pressure.

The size of the trap matters for the rate at which the pressure reaches equilibrium. In one embodiment, the trap and/or the fluid circuit between the trap and the source has a volume less than 1000, 500, 300, 150, 100, or 50 ml.

In some embodiments the negative pressure pump is configured to generate negative pressure of at least 100, 200, 300, 400, 500, or 700 mmHg as measured at the source (e.g., the air pump). Preferred pressures are greater than 300, 400, or 500 mmHg. The air flow rate can also be important to achieving good performance. In some embodiments, the free air flow rate of the negative pressure source (as measured at the source) can be less than 100, 85, 50, 25, 10, 5, 3 L/min, or greater than 0.1, 0.5, 1, 5, 10, 25, or 50 L/min or within a range of the foregoing endpoints. Preferred flow rates are less than 25, 10, or 5 L/min and greater than 0.1, 0.5, or 1 L/min.

In some embodiments the air flow rate selected to be relatively low such that the volume of air being removed from the tissue is relatively low to avoid vessel collapse. In a preferred embodiment, the ratio of negative pressure (mmHg) to the air flow rate (L/min) (free air flow as measured at the source) is relatively high compared to wall suction. The ratio can be greater than 10, 20, 50, 100, 200 or 400 HRU where HRU is hybrid resistance units and is a calculation of (mmHg)/(L/min).

In some embodiments, the suction control system is configured to set the pressure or respond to input from a user to set the pressure to a value within any of the foregoing pressures. The input can be digital or analog. The type of pump can be selected from the group consisting of diaphragm pump, rotary vane, scroll pump and the like.

In some embodiments, the pump is located in the proximal component. The pump can be located in a handle portion (i.e., gripping portion) of the device to minimize the size of the device. The handle is the region on the proximal portion where a user's hand would grip the device when the person's thumb is on the thumb actuator of the flexion assembly. Placing the pump in the proximal component can be advantageous to untether the endoscope from bedside tubing such as wall suction. This gives the clinician freedom of movement.

In an alternative embodiment, the pump is located in an external component (i.e., a unit external to the proximal and distal components of the endoscope). The external component is in fluid communication with the proximal component. Placing the pump in an external component can be advantageous for minimizing weight in the handheld portion.

The endoscope can also include an air valve, also referred to as an over-pressure valve. In some embodiments, the electrical processing unit actuates the air valve in response to a sensed pressure in the fluid circuit that is greater than a user selected pressure. The air valve can be a solenoid valve. In some embodiments, the air valve can be a mechanical pressure regulator.

In some embodiments, the endoscope includes a suction control system that includes the pressure sensor on a circuit board and a manifold mounted to the circuit board over the pressure sensor, the manifold providing fluid communication with an air outlet of the fluid trap and the air inlet to the negative pressure pump. The manifold may also be in fluid communication with the air valve for detecting over pressure. In some embodiments, a discrete pressure sensor may be used.

The portable endoscope includes a fluid trap port, the port including an inlet connector that is in fluid communication with the working channel and receives liquids and air from the working channel, and an outlet connector that is in fluid communication with the source of negative pressure. The inlet connector and the outlet connector may be tubing connectors.

The trap port may also include a filter in the fluid circuit between the outlet connector and the negative pressure pump. The filter can be a hydrophobic filter to hinder water in the trap from flowing out the air outlet. The filter may filter out biological material. For example, a 0.22 micron filter may be used.

The endoscope may also include an accessory port for inserting fluid in the working channel. The fluid circuit can include a fluid valve that prevents backflow of fluids towards the fluid trap or source of suction. The fluid valve may also prevent backflow of fluids to and/or out of the distal end when for, example, suction is stopped.

The endoscope includes a flexion control system including a thumb lever, a flexion wheel, and a plurality of steering wires. The flexion control system provides user control of the flexible tubing of the distal component and is used to steer the distal end of the endoscope.

The endoscopes of the invention include a light source and lens and typically an image sensor at the distal end for capturing images inside the body. The images are transmitted to the proximal component and either processed in the proximal component or image data is transmitted to an external processing unit and then displayed. The transmission can be wired or wireless. The clinician uses the displayed images to navigate the distal end of the endoscope through the body using the flexion control system and manual manipulating the proximal component of the endoscope (e.g., pushing and pulling or rotation).

Endoscopes vary in size and form to overcome numerous anatomical challenges from different regions of the body. Endoscopes may, for example, navigate tight spaces and tortuous channels. Endoscopes have demonstrated their utility in diagnosing and treating diseases and conditions of the lungs (bronchoscopes), upper digestive tract (endoscope), urethra and bladder (cystoscope), colon and rectum (colonoscope), abdomen and pelvic area (laparoscope), larynx (laryngoscope), and other regions of the body.

These endoscopes may, for example, include a long tube or insertion portion/tube connected to a handle. At the end or tip of the endoscope, there is a small camera, light source(s), and typically the end of an internal tube that may, for example, be part of a working channel. This working channel can serve multiple purposes including irrigation, introduction of medical instruments designed to collect samples, and suctioning of bodily fluids. Suctioning may, for example, be used to clear plugged passages, collect samples, and improve visualization.

Several exemplary embodiments of the endoscope and its components and other potentially advantageous features that may be applied more generally to endoscopes and/or components thereof will be depicted in the following description. Specific details, arrangements of components, and designs and diagrams that establish multiple modalities are presented to provide a comprehensive understanding and visualization of the embodiments.

The invention relates to technology involved in an endoscope with independent, robust, and advanced suctioning abilities. Various embodiments may, for example, generate suction without an external suction source. Some such embodiments may, for example, advantageously, reduce or eliminate patient exposure to external tubing and/or an external suction source. Embodiments that do not use external tubing and/or an external suction source may, for example, advantageously reduce an amount of maintenance, inspections, disinfection, and/or sterilization of such systems. This may, for example, advantageously save time and/or costs associated with each endoscopy procedure. Some embodiments may, for example, advantageously reduce the likelihood of a patient acquiring an infection and/or needing re-admission (e.g., by decreasing the number of components that may, for example, need to be sterile for each procedure).

In some embodiments, the endoscope may, for example, contain a built-in suction pump which may be used to independently suction fluid into a collection container. In exemplary embodiments, the built-in suction pump may, for example, be positioned in the fluid communication line relative to the collection container and the tip such that the built-in suction pump does not encounter a liquid being withdrawn (e.g., body fluid). For example, in some implementations, the built-in suction pump sucks air from the fluid collection container and the working channel to generate a negative pressure. The negative pressure may, for example, advantageously drive fluid movement from the tip to the fluid collection container in which the liquid may, for example, be stored. In implementations in which bodily fluids (e.g., viscous liquids, non-viscous liquids) do not pass through the built-in suction pump (e.g., indirectly by applying negative pressure indirectly through a second fluid such as air and/or directly by blocking the fluid from entering the pump), the likelihood of the pump clogging, suffering in performance, and/or failing may, for example, be minimized.

Some embodiments of the endoscope may, for example, enable connection to an external suction source to add further suctioning abilities. This can, for example, advantageously be used to create a more powerful suction, increase flow rate, provide a backup system in case the built-in suction pump fails, and/or allow fluid to bypass the collection container.

The endoscope may, for example, come in both wireless and/or cabled forms. In the cabled forms, power may, for example, be provided. In the cabled form, data may, for example, be transferred to and instructions received from an external display device through a cable.

In some embodiments (e.g., wireless forms), an internal battery may, for example, provide power to the endoscope. Data and instructions may, for example, be communicated wirelessly with an external display device.

Suction control systems herein may, for example, advantageously provide an endoscope in which the suction may, for example, be generated by at least one built-in suction pump, the external suction source, or both (e.g., as depicted and drawn). In some implementations, for example, a suction control interface may allow an operator to selectively apply one or more available suction sources.

Some embodiments of the invention relate to improving suction through improvements to geometry of the distal end of the working channel. For example, some embodiments of the endoscope are described as having a camera module assembly placed distally relative to the opening of the working channel. A larger working channel can be implemented with the larger opening compared to the working channel of a similarly sized insertion tube using a conventional tip.

In this embodiment, the working channel may, for example, have a different diameter within the insertion tube than within the articulating region. Such an endoscope may, for example, be advantageous over conventional endoscopes by allowing a larger tube to function as the working channel when not restricted in size by the articulating part. This may, for example, advantageously increase the suction flow rate of an endoscope with the same diameter insertion tube.

An embodiment of an endoscope also includes an insertion tube that that does not require a separate internal tube to form the working channel. Rather, the insertion tube can be used as the working channel. Such an endoscope may, for example, use the outer tube of the insertion portion as the working channel. Such an endoscope may, for example, be sealed in the articulating region by a sleeve or bending rubber. This may, for example, advantageously allow the full cross-sectional area of the insertion portion to be used, for example, for suctioning and insertion of instruments. This may, for example, advantageously increase the suction flow rate and size of instruments that may, for example, be passed through the endoscope. Such an endoscope may, for example, advantageously eliminate the need for the internal tube and reduce unused space within the insertion portion.

By way of example, but not in limitation, several configurations are provided in the following description. For purposes in this document, an external suction source and external suction system may, for example, be used interchangeably. These terms may, for example, include an external suction source, such as that provided from hospital wall suction. These terms may, for example, include a portable vacuum pump and/or an external suction source with a collection container as may, for example, be used to collect sputum and other bodily fluids in the clinical environment. For example, an external suction source interpretation may be used in some embodiments when fluid may, for example, bypass a mounted collection container. Unless otherwise specified, for purposes in this invention, the term “insertion tube” and “insertion portion” may be used interchangeably.

Some endoscopes may, for example, use a large external negative pressure suction source. This suction source may include the hospital wall suction and/or a portable large suction pump. In the case of a hospital wall suction source, multiple devices may be connected, thus drawing from the same suction source. In some instances, this can reduce and destabilize the suction flow rate of the endoscope.

In the case of the portable large suction pumps, they may, for example, be quite heavy and cumbersome to use, making them difficult to transport between clinical rooms. For both hospital wall suction and portable suction pumps, hospital staff may, for example, abide by strict and time-consuming protocols that may involve, for example, daily inspections, routine system maintenance, cleaning of the collection canisters, replacement of filters, and disinfection and sterilization of the suction system. In the case of portable suction pumps, they may, for example, be disassembled, sterilized, and reassembled before each use. Adherence to such protocols is critical to prevent hospital-acquired infections, but significantly increases the cost associated with each procedure. Furthermore, it reduces the number of patients that can receive care.

Additionally, current endoscopes may, for example, need tubes to connect to external suction sources. These external tubes may, for example, create obstacles in the clinical environment that can hamper the movement of clinicians. For example, numerous severe orthopedic injuries from tripping over the tubes have been reported in literature. The external tubes may, for example, tangle with tubes used for other devices in the hospitals and clinics. The tangled tubes can pull on the suction tube connected at the suction port on endoscopes, causing the suction tube to be disconnected and the endoscope to lose suctioning ability. Furthermore, the length of the tubes connecting to the external suction source may, for example, add more flow resistance, thereby reducing the suction flow rate.

Some endoscopes have a cable connection that provides power to the endoscope and is used to transmit image data to an external display device. Similar to the tubing, the cable may, for example, impede movement of healthcare professionals during a procedure and can tangle with tubes and other cables in the hospital and clinic. When the cable gets tangled, the cable can disconnect from the endoscope causing it to lose power and video to not be captured and displayed on the display device. Because of the drawbacks of the tubing and cable connections, procedures are prolonged and some clinicians are dissatisfied with current options.

Some endoscope tips are typically made such that they have a flat end with a light source, camera, and constricted, circular working channel in approximately the same plane. This constricted working channel reduces the suction flow rate, limits the size of instruments that may pass through an endoscope, and can interfere with the ability of the endoscope to suction highly viscous mucus plugs. One endoscope currently marketed contains a clamshell-shaped working channel on the tip that enables increased suctioning capabilities from its greater cross-sectional area.

Some endoscope working channels typically comprise a tube that runs from the tip through the inside of the insertion tube and into the handle. This tube may, for example, not occupy the maximum cross-section available for use as the working channel. This tube may, for example, have the same diameter along its entire length. The tube's outer diameter may, for example, be limited by restrictions encountered in the tip and the articulating region. By using the same diameter tube along the entirety of the insertion portion, the suction of an endoscope may, for example, not be optimized and may be inadequate for suctioning, for example, some highly viscous fluids or mucus plugs.

Some endoscopes include the ability to mount sampling containers that collect fluid from the procedure. In some cases, such as in a bronchoalveolar lavage (BAL), the fluid collected is later analyzed to determine, for example, if the patient has an abnormal condition, infection, or cancer.

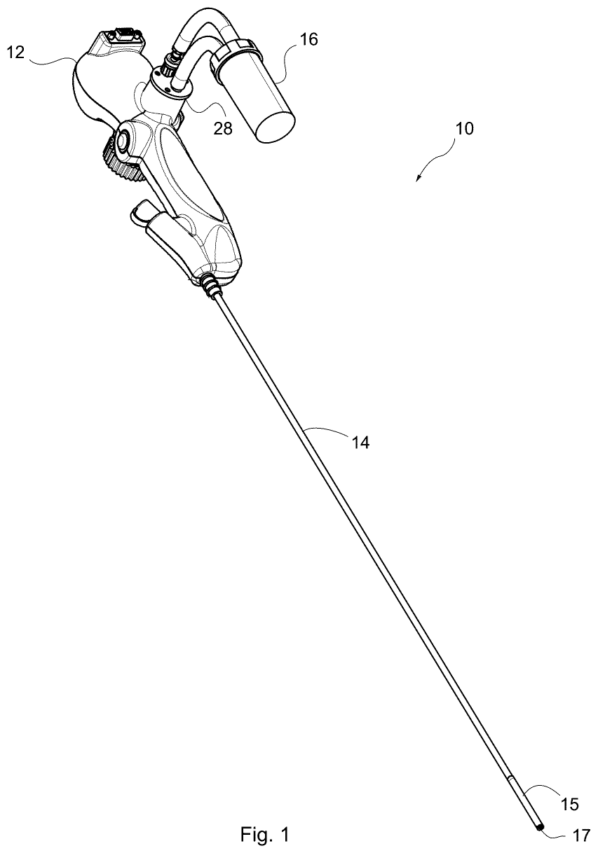

FIGS. 1-9 illustrates endoscopes according to examples of particular embodiments of the invention. All or a portion of the components illustrated in FIGS. 1-9 can be used alone or in combination with any of the other components of the embodiments of the invention described herein. With reference to FIG. 1, endoscope 10 is a bronchoscope with a proximal component 12 and distal component 14. A liquid trap 16 is connected to the proximal component 12 through port 28. Distal component 14 includes an articulating portion 15 with a camera at the distal end 17. Endoscope 10 may be a bronchoscope with insertion component 14 sized and configured for insertion to the airways of a lung.

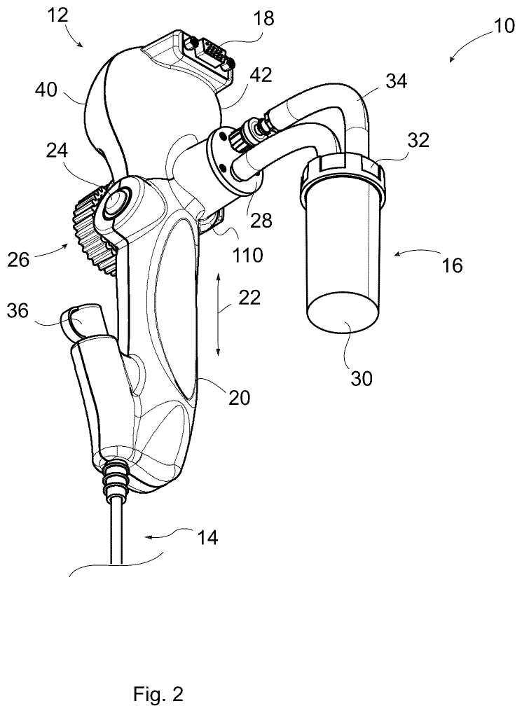

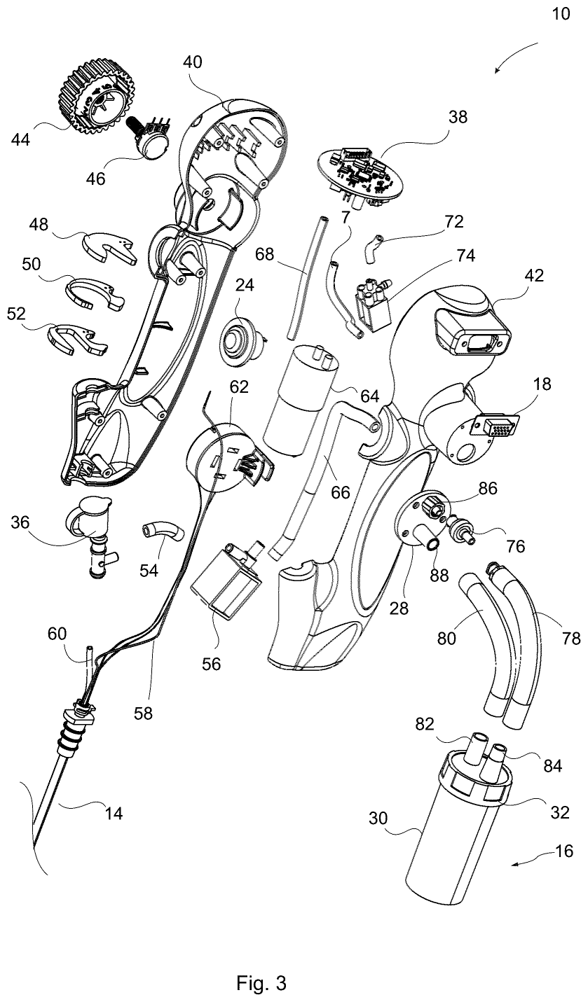

FIG. 2 illustrates the proximal component 12 of endoscope 10 in more detail. Proximal component 12 is formed from a housing (left housing 42 and right housing 40) that houses the internal components shown in the exploded view of FIG. 3. With reference again to FIG. 2, the proximal component includes a button 24 to turn on and off the suction to working channel of distal component 14. A pressure selector 26 is used to adjust a max pressure of the bronchoscope.

The housing 40 and 42 forms a handle 20 that extends axial a distance 22 for allowing a user to hold the device. The handle 20 positions the hand with the user's thumb on thumb actuator 110 and an index finger positioned to trigger button 24. Proximal component 12 also includes an electrical connector that connects endoscope 12 to external power, video processing, and/or image display. A liquid trap 16 is connected to a port 28 of proximal component 12 through tubing such as tubing 34. Liquid trap 16 includes a container 30 and a trap cap 74. Liquid trap 16 can be oriented downward (i.e., axially aligned with distance 22 of handle 20 such that in use gravity causes liquids to collect in container 30 while air is expelled through tubing 34).

FIG. 3 provides an exploded view of the parts of endoscope 10. FIG. 3 shows the main components of endoscope 10 with some wiring removed for clarity. Circuit board 38 includes electrical circuitry for sending and receiving signals and executing computer readable code. Circuit board 38 includes a processor, random access memory, and storage space for executing the code. The circuit 38 is electrically coupled to the pump 64, position sensor 46, air valve 74, fluid valve 56, button 24, and cable 58 for image data. Circuit board 38 is also electrically coupled to electrical connector 18 to receive power and transmit and/or receive data externally. For example, circuit 38 can process or transmit video images from camera 15 to an external display for viewing by a clinician operating endoscope 10. Circuit 38 can send and/or receive electrical signals to/from any of the foregoing components and use the signals to execute computer implemented methods on the circuit board. The inputs may be user input elements. For example, circuit board can receive input from button 24, or position sensor 46 to turn on or off pump 64, actuate a solenoid in fluid valve 56, or actuate a solenoid in air valve 74. Control knob 40 mechanically couples with position sensor 46 and can be rotated by a user to provide user input that sets a user input for the value for the max pressure of the system. Position sensor 46 can be a potentiometer or other type or rotation position sensor. The sensor can also be a different type of sensor such as rotary encoder, optical sensing component, magnetic, or the like.

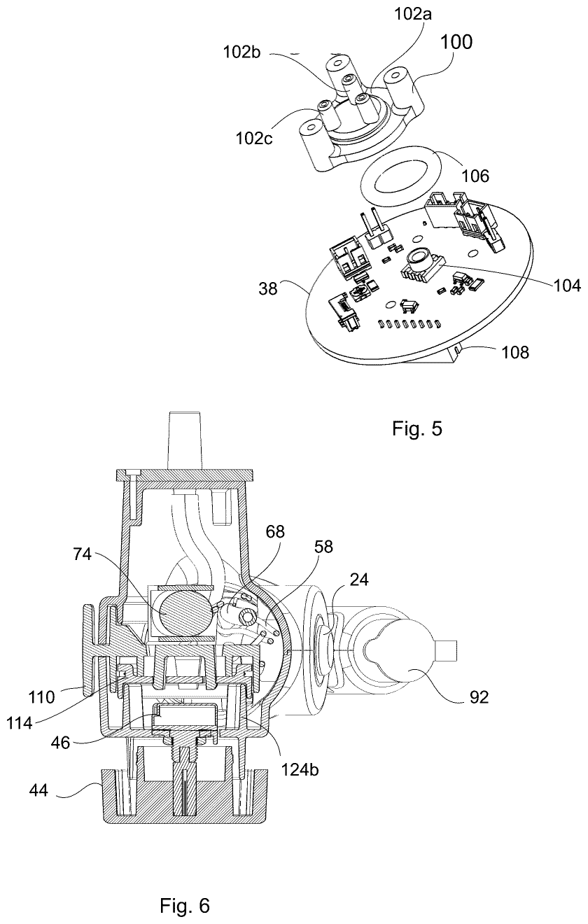

Rib 48, rib 50, and rib 52 are support structures that nestle in housing 40 and support pump 64. Ribs 48, 50, and 52 also provide a path for wires and tubing to pass through handle 20. Accessory port 36 connects to tubing 54, which connects to fluid valve 56. Fluid valve 56 also connects to tubing 66, which connects to port 28. Tubing 68 connects to pump 64 and manifold 100 (see FIG. 5). Tubing 72 connects to manifold 100 and air valve 74. Tubing 70 connects to the manifold 100 and an air port (not to be confused with a landing strip for airplanes) on port 28. Port 28 also have a connector 86 that couples with filter 76. Connector 86 can be a luer lock connector. Trap 16 includes container 30 and trap cap 32 that form a reservoir for separating liquid and air. Cap 32 includes an inlet 82 and an outlet 84, which couple with tubing 80 and tubing 78, respectively. Tubing 80 connects inlet 82 to connector 88 to form a fluid path to tubing 66 and fluid communication with working channel 60. Tubing 78 connects outlet 84 to filter 76 to form a fluid path to pump 64 and air valve 74.

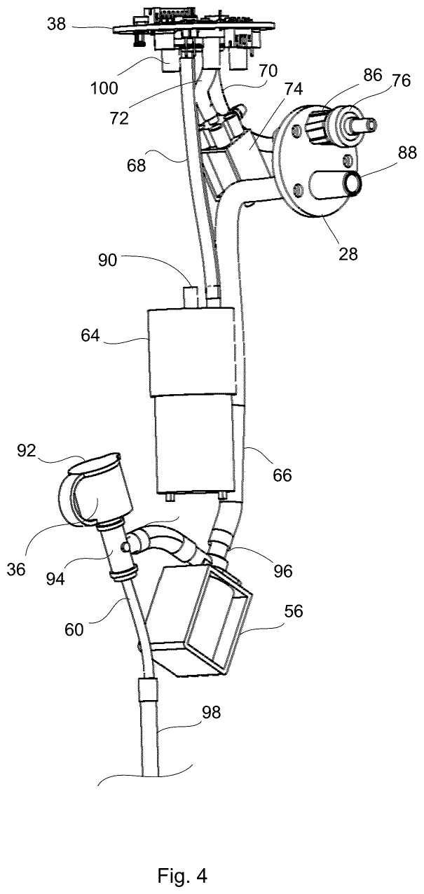

FIG. 4 illustrates the fluid circuit of the proximal component in more detail. The liquid trap has been removed to show connector 88, which connects the liquid trap to the fluid circuit of the proximal component. Negative pressure at port 88 creates negative pressure in tubing 66.

Accessory port 36 includes a valve for inserting tools into a working channel 60 that is positioned within flexible tubing 98. Accessory port 36 is connected to t-connector 94. T-connector 94 connects to working channel 60. T-connector 94 is also connected to tubing 54 and connector 96 of fluid valve 56. Valve 56 receives an on-off signal from circuit board 38. When circuit board 38 sends an “on” signal, a solenoid in 56 opens a valve in connector 96 and negative pressure in tubing 66 is transmitted through connector 96, tubing 54, connector 94 and into working channel 60. An “off” signal from circuit board 38 will cause the solenoid in fluid valve 56 to extend into connector 96 and block fluid flow, which cuts negative pressure to working channel 60 and turns pressure “off”. While valve 56 has been shown using a solenoid, other electrically controlled valves can be used or even mechanical fluid valves that do not require the use of the electrical circuitry.

With reference to FIGS. 4 and 5, pump 64 is a source of negative air pressure. Pump 64 is powered and controlled by circuit 38 and pumps air out outlet 90, releasing it to the internal cavity of proximal component 12, which is not hermetically sealed and leaks the air to the ambient. In an alternative embodiment, port 90 can have tubing (not shown) that releases outside of the housing of proximal component 12. Channeling the air to ambient can avoid noises from leaking. The speed of pump 64 creates a negative pressure at the inlet of pump 64. Tubing 68 connects to inlet 102a of manifold 100. Manifold 100 is attached to circuit board 38 and forms a seal over pressure sensor 104 using an o-ring 106. The space between circuit board 38 and manifold 100 creates a manifold chamber where negative pressure from pump 64 equilibrates to a pressure with tubes 68, 70, and 72, which lead to pump 64, trap 16, and air valve 74. Pressure sensor 104 is electrically mounted to circuit board 38 and provides pressure readings to the computing system. The pressure readings can be used to compare the measured pressure to the target pressure and adjust power delivered to pump 64 accordingly. The pressure readings can be used to control the air valve 74 to lower pressure in the fluid circuit by opening a solenoid valve and allowing ambient air into the fluid circuit, which reduces pressure. Pressure sensor 104 can sense the change in pressure and apply a signal to maintain the valve more closed or more open as needed to achieve the desired value for the pressure. The negative pressure from pump 64 can be transmitted to working channel 60. Negative pressure is transferred to manifold 100 and then to tubing 70 and then to filter 76, then to trap 16. Consequently, the negative air pressure from pump 64 is applied to liquid trap 16. Because liquid trap 16 traps liquid fluid in its container 30, liquids do not travel into or through port 84 or tubing 70. Thus, pressure sensor 104 can have accurate pressure readings even when there is liquid occluding working channel 60. Filter 76 can be a hydrophobic filter to further ensure that only gases flow through filter 76.

With reference to FIGS. 1-5 the fluid trap is described in greater detail. The fluid trap may include a mountable liquid collection container 30 which may, for example, store fluids suctioned during an endoscopic procedure. The fluid collection container 30 includes, in this example, a fluid collection reservoir for storing patient sample. The collection container 30 may include markings for measuring the amount of fluid in the container 30. The container may have a volume of at least 15, 30, or 60 ml and less than 300, 150, or 60 ml or a range of the foregoing endpoints. The foregoing volumes are useful for collecting patient samples. However, in other embodiments, the endoscope may have a liquid trap with much smaller volumes e.g., less than 10, 5, or 1 ml where collecting a patient sample is not necessary.

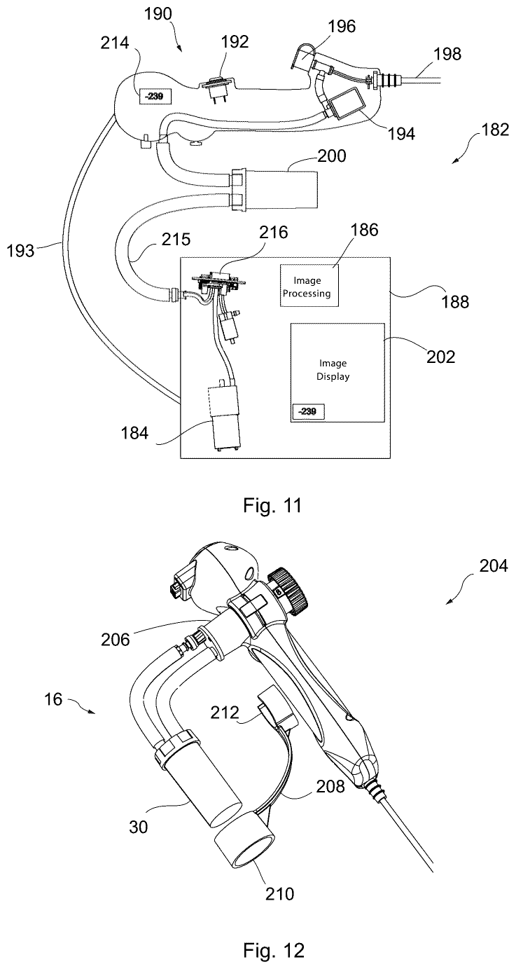

Liquid traps according to the invention may be attached to the proximal component by various means including screw connectors, snaps, or tubing connectors. In some embodiments it is beneficial to seal the liquid trap to maintain pressure and prevent noise from leaks. The seal can be created by a screw mount, a polymeric seal such as an o-ring, or polymeric tubing that forms a seal with a connector. FIGS. 1-5 show an embodiment where the liquid trap is mounted using tubing connectors. In this embodiment it can be useful to use a mounting bracket (see e.g., FIG. 12).

The liquid trap can use gravity to separate liquid from air and trap the liquid in the container. This is achieved by placing inlet 82 and outlet 84 in the cap of trap 16. Liquids from working channel 60 enter the trap at the top and fall to the bottom of container 30. Gases enter the same inlet 82, but flow to outlet 84 due to the negative pressure, thereby separating liquid and air.

The container 30 may be made from any material including but not limited to glass and/or a translucent and/or transparent thermoplastic suitable for injection molding (e.g., polypropylene, polycarbonate). The trap cap 32 may, for example, be made from a strong thermoplastic suitable for injection molding (e.g., ABS, ABS-PC, acrylic, polypropylene, polyethylene, polycarbonate).

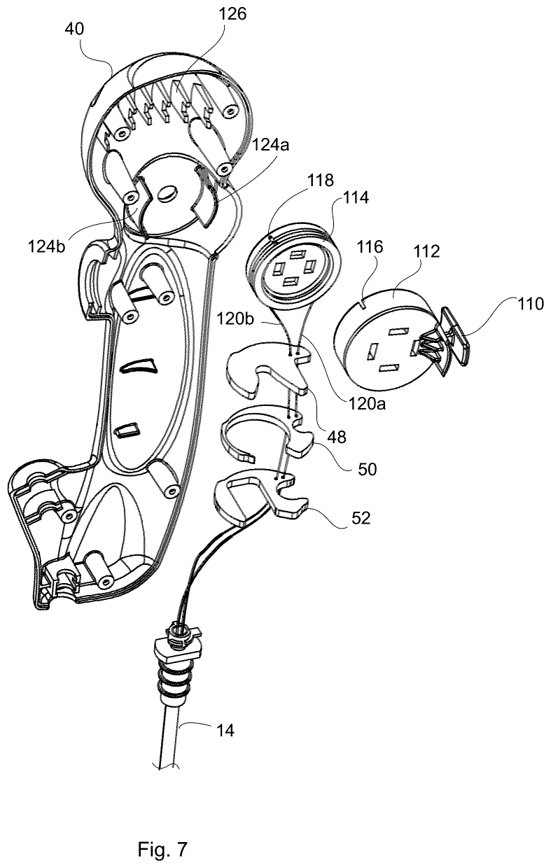

With reference to FIG. 7, the flexion components are described in more detail. The flexion components may, for example, be used in an endoscope to control the bending and deflection of the articulating portion 15 and distal end 17 that houses the camera. This may, for example, advantageously enable the flexible tubing 98 of the endoscope to navigate through narrow and/or tortuous passages.

FIGS. 6 and 7 illustrates an example flexion control interface configured to mate with housing 40. Housing 40 can be configured to securely mount wheel 114 while still providing a path through which tubing and wires can pass and electrical components can be mounted in axial alignment with the flexion wheel 114. For example, housing 40 includes two curved walls 124a and 124b that receive flexion wheel 114. Curved walls 124 provide a space for position sensor 46. The discontinuity in walls 124 allows wires for the position sensor to reach the circuit board 38 which sits in mounts 126. On an opposite side of the flexion device from the position sensor sits air valve 74. As shown in FIG. 6, tubing 68 and wires 58 pass through the space provided by an open mounting configuration with discontinuous mounting walls to provide space for the tubing and wires to pass under or over the flexion system. Ribs 48, 50 and 52 also facilitate organizing the steering wires 120 and tubing and electrical wires. For instance, ribs 48, 50, and 52 include lateral notches that are configured to accommodate tubing and wires. Example of tubing and wires accommodated by the ribs are example tubing 68 and wire 58 illustrated in FIG. 6.

FIG. 7 illustrates the components of the flexion system. In the depicted example, the steering wires 120a and 120b (collectively wires 120) each have a crimp/shell 118 at their end. The crimp/shell may, for example, be coupled to the steering wires 120 through soldering or mechanical means. The crimp/shell may, for example, be larger than the diameter of the steering wires 120. The crimp/shell may, for example, sit in a cavity 116 formed when the second wheel 114 is connected. The crimp/shell resting in the cavity may, for example, advantageously lock the steering wires 120 in their positions with respect to the wheels 114. The steering wires 120 may, for example, be fixed to the wheels 114 through other means such as, for example, plastic welding, ultrasonic welding, and adhesives. Barrel adjusters may, for example, be implemented to advantageously adjust tension on the steering wires 120. As depicted in this illustrative embodiment, the steering wires reside in a groove along the circumference of the wheel 114.

When the user moves the thumb actuator 110 clockwise, this may, for example, cause the flexion control interface to pull on the steering wire 120b causing a corresponding clockwise deflection of the end 117. When the user moves the thumb actuator 110 counterclockwise, this may, for example, cause the flexion control interface to pull on the steering wire 120a causing a corresponding counterclockwise deflection of the distal end 117.

In some implementations, the components described in the flexion control interface may, for example, be made from thermoplastics which are strong and can be easily injection molded (e.g., ABS, ABS-PC).

Internal tubing of the endoscope of the invention may, for example, comprise flexible, thin wall plastic and/or elastomeric tube(s). The tubing may, for example, have a small bend radius and sufficient rigidity to not collapse under a predetermined suction pressure (e.g., −500 mmHg). Such tubing may, by way of example and not limitation, be made from polyvinyl chloride (PVC), polyurethane (PU), polydimethylsiloxane (PDMS), and/or polytetrafluoroethylene (PTFE).

In this example, steering wires 120 run from the distal end of the distal component 14 near the distal end 15 to the flexion control interface. The flexion control interface may, for example, freely rotate within the handle 20 but be constrained in its position. Within the handle 20, the steering wires 120 may, for example, be guided by ribs 48, 50, and 52 that separate and sandwich the steering wires 120 between the ribs and the right half 44. This may, for example, advantageously serve to case assembly, ensure the correct tension is on the steering wires 120, and/or prevent the steering wires 120 from crossing. Barrel adjusters may, for example, be implemented to advantageously adjust tension on the steering wires 120. In some examples, the ribs may be made from a strong thermoplastic suitable for injection molding (e.g., ABS, ABS-PC).

Barrel adjusters may, for example, be implemented to advantageously adjust tension on the steering wires 81 and 82.

As depicted, there may, for example, be at least one built-in suction pump held within the handle. Its position is stabilized and/or constrained by ribs on both the right half 40 and the left half 42. The built-in suction pump may, for example, include a pump that is capable of pumping/suctioning air and/or liquid (e.g., a diaphragm pump).

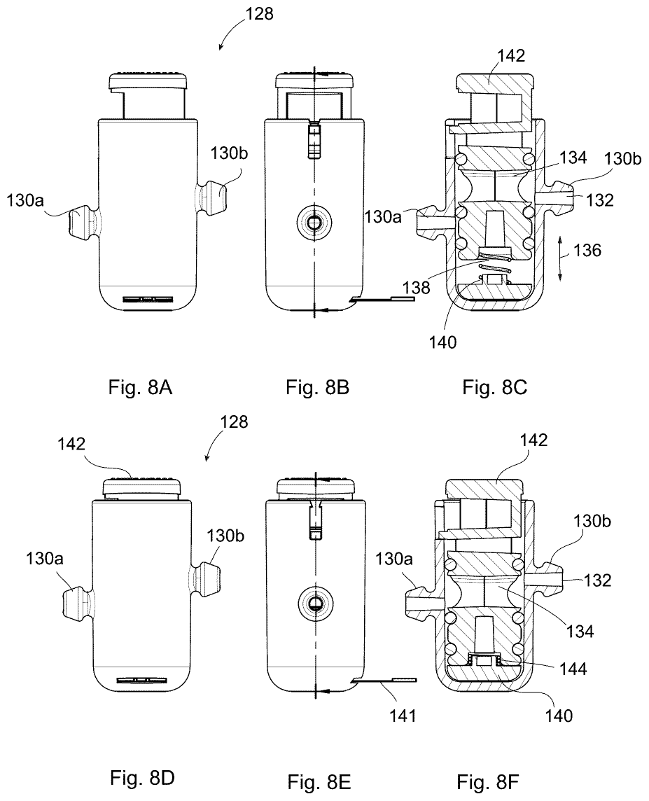

FIGS. 8A-8F illustrate a suction control interface 128 for controlling suction applied to an endoscope. The suction control interface 128 includes an outer cylinder with a first port 130a and a second port 130b, each having an opening 132. The interface 128 includes an actuator 142 with a plunger body. The plunger body has an inner channel 134 that moves down when the actuator is pressed and up when the actuator is released and coil 144 biases the actuator 142 to the up or off position as shown in FIGS. 8A-8C. When actuator 142 is pressed downward, the inner channel 134 moves down and is aligned with the openings of ports 130a and 130b and fluid can flow through the ports and inter channel. This aspect of suction control interface 128 can turn pressure on and off mechanically by forming the path through suction control interface 128.

Control interface 128 also includes an electrical actuator. Below the plunger body is a force sensitive resistor 140 that senses force from the plunger. When the plunger is in the up position as in FIG. 8C the spring force on the force sensitive material 140 is low. As the plunger is pressed downward as shown in FIG. 8F the spring 144 is compressed and more force is applied to force sensitive material 140. This force is sensed and provided to the circuitry of the endoscope through an electrical connection. The electrical connection can be made with contact 141.

Those skilled in the art will recognize that other suction control interfaces according to the invention may be provided for either mechanically blocking and unblocking the working channel fluid flow or for sensing a pressure applied to the suction control interface and using a computer processor to regulate a valve or pump using the sensed pressure.

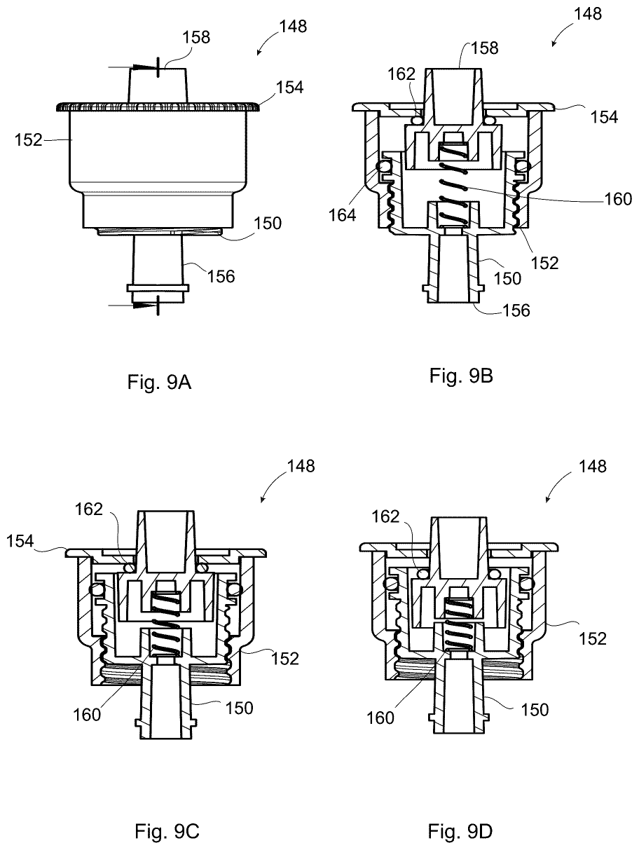

FIG. 9A-9D illustrate an example embodiment of negative pressure regulator 148 that can be used in an endoscope according to one embodiment of the invention. The negative pressure regulator 148 can be used to control the pressure in an endoscope from a source of negative pressure. One advantage of a negative pressure regulator is that it can provide regulation of the pressure mechanically without the need for an electrical processing unit, thereby avoiding some of the complexities of using electrical circuits. In FIG. 9A, the regulator 148 includes a male component 150. The male component 150, may by way of example and not of limitation, be threaded. The regulator 148 may, for example, have a female component 152 into which male component 150 is threaded. The female component 152 may, by way of example and not of limitation, be threaded. Regulator 148 may, for example, have an interface piece 154 that a user can grip to turn the regulator. The interface piece 154 of regulator 148 may have an aperture. The aperture may, for example, be placed in the center of the interface piece 154. The regulator 148 may, for example, have a center button 158. The center button 158 may, for example, be sized appropriately for fitting through an aperture in the interface piece 154. The regulator 148 may, for example, have a seal 162 that may, by way of example and not of limitation, prevent air leakage between the male component 152 and female component 150. The regulator 148 may, for example, have a seal 162 that may, by way of example and not of limitation, prevent air leakage between the interface piece 154 and the center button 158. The regulator 148 may, for example, have a spring 160. The interface piece 154 may, by way of example and not of limitation, be adhered to and/or sealed with the female component 150. Therefore, movement of the interface piece 154 may, for example, cause movement of the female component 150. The example parts of the regulator 148 aside from the spring 160 may, by way of example and not of limitation, be made from metal and/or thermoplastics suitable for injection molding. The seals 162 and 164 may, by way of example and not of limitation, be made from a rubbery and/or soft elastomer such as PU, PDMS, or Viton. FIGS. 9C and 9D demonstrate an example embodiment of the regulator in a few different example states of operation. In FIG. 9B, the regulator 148 is in a retracted state. In the retracted state, the female component 152 may, for example, be located higher up relative to the male component 150. In the retracted state, the spring 162 may, for example, be under little to no compression. When the spring 160 is under little to no compression, the suction force required to compress the spring 160 further is less than if the spring 160 was under greater compressive strain. Suction force generated by, for example, negative pressure in the fluid line that the regulator 148 may, for example, be connected to may, for example, exceed that required to compress the spring 160 further than its established length determined by, for example, the position of the male component 150 with respect to the female component 152. When this occurs, the seal 162 may, for example, move with the center button 158 and may, for example, open sufficient space between the seal 162 and the interface piece 154 to, for example, enable an air leak. The suction force may, for example, be directly correlated to the negative pressure or suction pressure and the size of the aperture in, for example, an interface piece 154 of regulator 148. As a result of its operation, the regulator 148 may, for example, advantageously ensure the suction pressure does not exceed a value determined by the compression of spring 160 and correspondingly, the position of the female component 152 with respect to the male component 150.

In FIG. 9C, the regulator 148 is in a further compressed state than that shown in FIG. 9B. This may, for example, be achieved by movement of the interface piece 154 which correspondingly may, by way of example and not of limitation, cause movement of the female component 152 with respect to the male component 150. The example embodiment of the regulator 148 depicted in FIGS. 9A to 9D has a threaded male component 150 and a threaded female component 152 which means that turning the interface piece 154 clockwise may, for example, cause the female component 152 to move further down the male component 150. Meanwhile, turning the interface piece 154 counterclockwise may, for example, cause the female component 152 to move further up the male component 150. In the further compressed state, the female component 152 may, for example, be located further down relative to the male component 150. In the state shown in FIG. 9C, the spring 37 of the NPR 30 may, for example, be further compressed than the state shown in FIG. 9B. As a result, the suction force and corresponding negative pressure required to further compress the spring 160 and create an air leak between the seal 162 and the interface piece 154 may, for example, be greater.

In FIG. 9D, the example regulator 148 is in a state where the center button 158 is pressed. When the center button 31 is pressed, the seal 162 may, for example, move with the center button 31 and may, for example, open sufficient space between the seal 162 and the interface piece 154 to, for example, enable an air leak. This may, for example, cause the fluid communication line connected to the regulator 148 to equalize with the outside environment, thereby eliminating the negative pressure gradient.

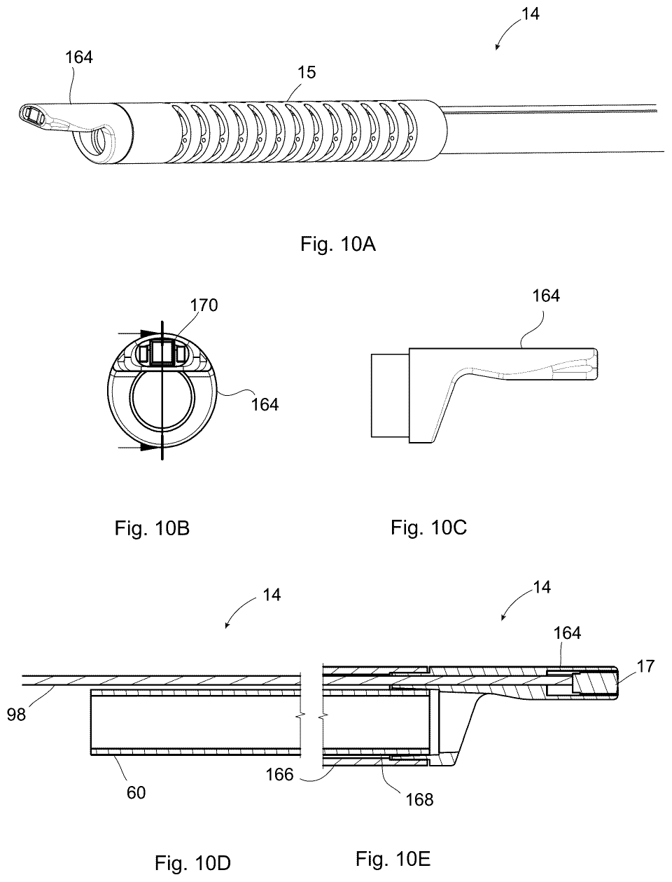

FIGS. 10A-10E illustrate an example of the insertion portion 14. As can be seen in FIGS. 10A-10E, the tip 164 is at the distal end of the insertion portion 14. In FIG. 10D, a section view within the insertion portion 14 can be seen. In the more proximal section view illustrated in FIG. 10D, there is an outer tube 98. In the more distal section view illustrated in FIG. 10E, a sleeve 166 may, for example, surround the articulating part 15. Within the articulating part 15, of FIG. 10A, there may, by way of example and not limitation, steering wires and the working channel 60. The steering wires may, by way of example and not limitation, each be within a thin-wall lubricous tube to promote case of sliding within the insertion portion 14. Not shown in FIGS. 10A-10E are electrical wires which may, for example, supply power and communicate data with the camera module assembly 170. In this example, these may, for example, be disposed outside the working channel 60, but within the outer tube 98 and the articulating part 15.

The outer tube 98 may, for example, be a wire reinforced plastic or thermoplastic elastomer tube. The wire reinforcement may, for example, advantageously add structural rigidity (e.g., ensuring that the articulating part 15 encounters the most bending). The wire reinforcement may, for example, provide shielding from electromagnetic interference (EMI).

As depicted, outer tube 98 may, for example, run up to the articulating part 15. In this example, the articulating part 15 is held in its position in two ways. First, it may be held in position by the sleeve 166 which may, for example, cover the articulating part 15 and the distal part of the outer tube 98. Second, the steering wires may, for example, be coupled (e.g., soldered, welded, adhered, mechanically locked) onto the distal end of the articulating part 15 and hold the end of the articulating part 15 at a fixed length from the flexion control interface to be described.

The sleeve 166 may, for example, be made of heat shrinkable and/or flexible material like FEP or PTFE tubing. The internal tubing of the working channel 60 may, by way of example and not limitation, be a flexible, thin wall plastic and/or elastomeric tube with a small bend radius and sufficient rigidity to not collapse under a predetermined suction pressure (e.g., −500 mmHg). Such a working channel 60 may, by way of example and not limitation, have tubing made from polyvinyl chloride (PVC), polyurethane (PU), polydimethylsiloxane (PDMS), and/or polytetrafluoroethylene (PTFE).

In some implementations, the articulating part 15 may be made from a thermoplastic elastomer and/or a chain of metal pieces which snap together in a series. This may, for example, advantageously allow the articulating part 15 to bend substantially.

FIG. 11 describes an alternative embodiment of the invention where the endoscope comprises a system 182 where the negative pressure pump 184 is located in an external component 188. The external component may be electrically coupled to a proximal component 190 through cable 193. Proximal component 190 also has a fluid connection to proximal component 190 through trap 200. Trap 200 has a first tube 215 connecting to the source of pressure in the external component 188. Tube 215 is fluidly coupled to the pump 184 and transfers the negative pressure from pump 184 to liquid trap 200. Negative pressure in liquid trap 200 is transmitted to tubing 218 for suctioning fluids from tissue through the working channel into the trap for separation into liquids and air.

The liquid trap 200 may have any of the features described with respect to other embodiments of the invention. The trap 200 may snap to or screw onto a portion of housing 188.

Proximal component 190 may include any number of user interfaces for receiving input from a user to control on off state and pressure. For example, proximal component 190 may have an on-off switch 102 and/or a fluid valve 194 for shutting off suction when suction is not desired or when the accessory port 196 is being used.

The external component 188 of the endoscope may include any number of components including housing, connectors, filters, power supply, cables, tubing, and valves for providing a source of suction and regulating the suction. The external component may have pressure control circuit board 216 with the computer circuitry and software for controlling pressure. The external component may also have image processing 186 for receiving camera data and providing it to an image display 202. The pressure control circuit 216 and/or the image processing 186 can provide a pressure reading to external display 202 or to a display on proximal component 190. Pressure control circuitry 216 can have any of the features of circuitry 38 described herein.

Circuitry 216 can, in combination with computer implemented instructions, provide a user interface for operating external component 216. The system can include configuration and operation menus for setting and manipulating the pressure applied to the working channel. The menus and configurations can be display through a GUI on display 202 in addition to the image display from the camera that observes the tissue at the distal end. Example settings that can be adjusted include a max pressure and/or a min pressure and/or a flow rate or value for HRU.

Another embodiment of an invention disclosed herein relates to an endoscope 204 that is configured to receive a mountable sample collection container 30. The container 30 can form part of a liquid trap 16 as described above with regard to FIG. 1. The endoscope 204 includes a bracket 208 that is configured to rotatably snap to a mounting point 206 on endoscope 204. The bracket 208 forms an arc between snap connector 212 and sample holder 210. Snap connector 212 is configured to snap onto mounting point 206 and rotate about the mount point. Sample holder 210 is configured to slide onto container 30 and hold container in a desired position. The rotatable connection between mount 206 and snap 212 allows the user to keep the container 30 in the correct orientation when endoscope 204 is oriented by the clinician.

The present invention also includes methods for using the endoscopes and computer program products that implement various aspects of the invention. Methods of use include using any of the foregoing endoscopes to perform a procedure. The method can include all or a portion of the following: 1. providing an endoscope as described herein. 2. Powering the endoscope to an operational mode in which the input selection elements are available and/or the negative pressure pump is applying pressure. 3. Inserting the endoscope into tissue of a patient (e.g., into the lungs of a patient). 4. Actuating a user input element to cause suction in the working channel of the endoscope with the endoscope inserted into the patient 5. Modulating the negative pressure of the scope one or more times while the insertion tube is inserted into the patient, the modulation including receiving user input from one or more user input elements. 6. Using an accessory port on the endoscope to inject a liquid into the patient (e.g., inject saline or anesthetic into the patient) 7. Collecting liquid from the patient using the suction applied to the working channel (e.g., collecting a sample in a collection chamber of a fluid trap. 8. Withdrawing the insertion tube from the patient.

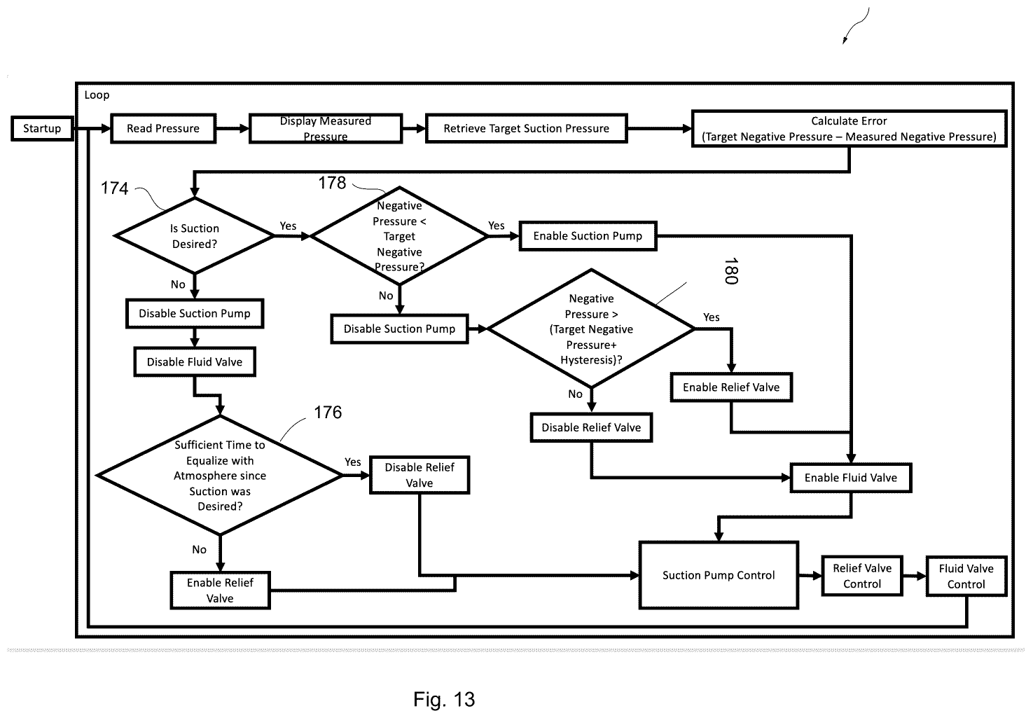

FIG. 13 is a flow diagram 172 of the logic of a computer program product for operating the electronics of one version of the invention. The computer program product and computing hardware are configured to startup a computer program product and then perform one or more of the following: After startup, the computing system reads pressure from one or more pressure sensors and optionally display the measured pressure either on an external display or a display mounted on the proximal component of the endoscope. The computing system then retrieves a target suction pressure from storage or memory and calculates an error by subtracting the measured negative pressure from the target negative pressure. The first determination is whether suction is desired. This can be derived from user input from an on-off button or a user interface or state of the device that indicates a desire for suction. If suction is not desired (i.e., block 174 is no), the system disables the suction pump and disables the fluid valve, and then determines if sufficient time has occurred to equalize with the atmosphere. If the answer is no, then the air valve is actuated to serve as a relief valve. If the answer is yes, then the relief valve is disabled and the suction pump is maintained in an off state.

If Suction is desired, (i.e., the logic of block 174 is yes), then the system goes to block 178 and calculates if negative pressure is less than target pressure. If block 178 is yes, then the pump is enabled and the fluid valve is enabled and the system progresses to using suction pump controls including using the pump, relief valve, and/or fluid valve to control pressure and/or turn pressure on and off.

If negative pressure is not less than target pressure (block 178 is no), the system disables the suction pump and determines if negative pressure is greater than the target negative pressure (block 180). If block 180 is no then the relief valve is disabled and the fluid valve enabled and the logic passes to suction pump control. If block 180 is yes (i.e., negative pressure is greater than the target), then the relief valve is actuated and the fluid valve enabled and control passed to the suction pump control.

In various embodiments, some bypass circuits implementations may be controlled in response to signals from analog or digital components, which may be discrete, integrated, or a combination of each. Some embodiments may include programmed, programmable devices, or some combination thereof (e.g., PLAs, PLDs, ASICs, microcontroller, microprocessor), and may include one or more data stores (e.g., cell, register, block, page) that provide single or multi-level digital data storage capability, and which may be volatile, non-volatile, or some combination thereof. Some control functions may be implemented in hardware, software, firmware, or a combination of any of them.

Computer program products may contain a set of instructions that, when executed by a processor device, cause the processor to perform prescribed functions. These functions may be performed in conjunction with controlled devices in operable communication with the processor. Computer program products, which may include software, may be stored in a data store tangibly embedded on a storage medium, such as an electronic, magnetic, or rotating storage device, and may be fixed or removable (e.g., hard disk, floppy disk, thumb drive, CD, DVD).

Although an example of a system, which may be portable, has been described with reference to the above figures, other implementations may be deployed in other processing applications, such as desktop and networked environments.

Temporary auxiliary energy inputs may be received, for example, from chargeable or single use batteries, which may enable use in portable or remote applications. Some embodiments may operate with other DC voltage sources, such as a 9V (nominal) batteries, for example. Alternating current (AC) inputs, which may be provided, for example from a 50/60 Hz power port, or from a portable electric generator, may be received via a rectifier and appropriate scaling. Provision for AC (e.g., sine wave, square wave, triangular wave) inputs may include a line frequency transformer to provide voltage step-up, voltage step-down, and/or isolation.

Although particular features of an architecture have been described, other features may be incorporated to improve performance. For example, caching (e.g., L1, L2, . . . ) techniques may be used. Random access memory may be included, for example, to provide scratch pad memory and or to load executable code or parameter information stored for use during runtime operations. Other hardware and software may be provided to perform operations, such as network or other communications using one or more protocols, wireless (e.g., infrared) communications, stored operational energy and power supplies (e.g., batteries), switching and/or linear power supply circuits, software maintenance (e.g., self-test, upgrades), and the like. One or more communication interfaces may be provided in support of data storage and related operations.

Some systems may be implemented as a computer system that can be used with various implementations. For example, various implementations may include digital circuitry, analog circuitry, computer hardware, firmware, software, or combinations thereof. Apparatus can be implemented in a computer program product tangibly embodied in an information carrier, e.g., in a machine-readable storage device, for execution by a programmable processor; and methods can be performed by a programmable processor executing a program of instructions to perform functions of various embodiments by operating on input data and generating an output. Various embodiments can be implemented advantageously in one or more computer programs that are executable on a programmable system including at least one programmable processor coupled to receive data and instructions from, and to transmit data and instructions to, a data storage system, at least one input device, and/or at least one output device. A computer program is a set of instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

Suitable processors for the execution of a program of instructions include, by way of example, both general and special purpose microprocessors, which may include a single processor or one of multiple processors of any kind of computer. Generally, a processor will receive instructions and data from a read-only memory or a random-access memory or both. The essential elements of a computer are a processor for executing instructions and one or more memories for storing instructions and data. Generally, a computer will also include, or be operatively coupled to communicate with, one or more mass storage devices for storing data files; such devices include magnetic disks, such as internal hard disks and removable disks; magneto-optical disks; and optical disks. Storage devices suitable for tangibly embodying computer program instructions and data include all forms of non-volatile memory, including, by way of example, semiconductor memory devices, such as EPROM, EEPROM, and flash memory devices; magnetic disks, such as internal hard disks and removable disks; magneto-optical disks; and CD-ROM and DVD-ROM disks. The processor and the memory can be supplemented by, or incorporated in, ASICs (application specific integrated circuits).

In some implementations, each system may be programmed with the same or similar information and/or initialized with substantially identical information stored in volatile and/or nonvolatile memory. For example, one data interface may be configured to perform auto configuration, auto download, and/or auto update functions when coupled to an appropriate host device, such as a desktop computer or a server.

In some implementations, one or more user-interface features may be custom configured to perform specific functions. Various embodiments may be implemented in a computer system that includes a graphical user interface and/or an Internet browser. To provide for interaction with a user, some implementations may be implemented on a computer having a display device. The display device may, for example, include an LED (light-emitting diode) display. In some implementations, a display device may, for example, include a CRT (cathode ray tube). In some implementations, a display device may include, for example, an LCD (liquid crystal display). A display device (e.g., monitor) may, for example, be used for displaying information to the user.

Some implementations may, for example, include a keyboard and/or pointing device (e.g., mouse, trackpad, trackball, joystick), such as by which the user can provide input to the computer.

In various implementations, the system may communicate using suitable communication methods, equipment, and techniques. For example, the system may communicate with compatible devices (e.g., devices capable of transferring data to and/or from the system) using point-to-point communication in which a message is transported directly from the source to the receiver over a dedicated physical link (e.g., fiber optic link, point-to-point wiring, daisy-chain). The components of the system may exchange information by any form or medium of analog or digital data communication, including packet-based messages on a communication network. Examples of communication networks include, e.g., a LAN (local area network), a WAN (wide area network), MAN (metropolitan area network), wireless and/or optical networks, the computers and networks forming the Internet, or some combination thereof. Other implementations may transport messages by broadcasting to all or substantially all devices that are coupled together by a communication network, for example, by using omni-directional radio frequency (RF) signals. Still other implementations may transport messages characterized by high directivity, such as RF signals transmitted using directional (i.e., narrow beam) antennas or infrared signals that may optionally be used with focusing optics. Still other implementations are possible using appropriate interfaces and protocols such as, by way of example and not intended to be limiting, USB 2.0, Firewire, ATA/IDE, RS-232, RS-422, RS-485, 802.11 a/b/g, Wi-Fi, Ethernet, IrDA, FDDI (fiber distributed data interface), token-ring networks, multiplexing techniques based on frequency, time, or code division, or some combination thereof. Some implementations may optionally incorporate features such as error checking and correction (ECC) for data integrity, or security measures, such as encryption (e.g., WEP) and password protection.

In various embodiments, the computer system may include Internet of Things (IoT) devices. IoT devices may include objects embedded with electronics, software, sensors, actuators, and network connectivity which enable these objects to collect and exchange data. IoT devices may be in-use with wired or wireless devices by sending data through an interface to another device. IoT devices may collect useful data and then autonomously flow the data between other devices.

Various examples of modules may be implemented using circuitry, including various electronic hardware. By way of example and not limitation, the hardware may include transistors, resistors, capacitors, switches, integrated circuits, other modules, or some combination thereof. In various examples, the modules may include analog logic, digital logic, discrete components, traces and/or memory circuits fabricated on a silicon substrate including various integrated circuits (e.g., FPGAs, ASICs), or some combination thereof. In some embodiments, the module(s) may involve execution of preprogrammed instructions, software executed by a processor, or some combination thereof. For example, various modules may involve both hardware and software.

Reference throughout this specification to “an example” means that a particular feature, structure, or characteristic described in connection with the example is included in at least one embodiment of the present disclosure. Thus, appearances of the phrase “in an example” in various places throughout this specification are not necessarily all referring to the same embodiment.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on its presentation in a common group without indications to the contrary. In addition, various embodiments and examples of the present disclosure may be referred to herein along with alternatives for the various components thereof. It is understood that such embodiments, examples, and alternatives are not to be construed as de facto equivalents of one another but are to be considered as separate and autonomous representations of the present disclosure.

Example of particular embodiments of the invention include, but are not limited to:

A portable endoscope with controlled suction, the endoscope comprising a proximal component and a distal component, the proximal component including a handle and the distal component comprising a flexible insertion tube extending from the proximal component to a distal end, the insertion tube including an internal working channel extending from the distal end to the proximal component; a powered suction system including a fluid circuit in fluid communication with the internal working channel of the proximal component, the powered suction system providing suction to the distal end, the powered suction system including, a source of negative pressure from a pump; a liquid trap in the fluid circuit, the liquid trap in fluid communication with the source of negative pressure; a suction control system comprising at least one user input element, the suction control system configured to use a sensed pressure to manipulate the negative pressure applied to the working channel. Manipulating includes turning the pressure on and off or changing the pressure or changing the max or min pressure.

In another example, the powered suction system includes a pressure sensor configured to sense the negative air pressure applied to the trap and the suction control system includes an electrical processing unit electrically coupled to the pressure sensor and providing the sensed pressure.

In another example the suction control system includes a mechanically actuated negative pressure release valve.

In another example, the proximal component includes one or more user input elements that provide input to the suction control system to selectively adjust the regulated negative pressure applied to the working channel of the distal component, wherein the electrical processing unit is responsive to the user input for regulating suction pressure.

In another example, a portable endoscope, includes a proximal component and a distal component, the proximal component including a handle and the distal component comprising a flexible insertion tube extending from the proximal component to a distal end, the insertion tube including an internal working channel extending from the distal end to the proximal component; a powered suction system including a fluid circuit in fluid communication with the internal working channel of the proximal component, the powered suction system providing suction to the distal end, the powered suction system including, a negative pressure pump positioned in the proximal component; a liquid trap in the fluid circuit, the liquid trap in fluid communication with the pump; a suction control system comprising at least one user input element, the suction control system configured modulate the negative pressure applied to the working channel.

Any of the foregoing examples can be combined with any of the features of the embodiments of the invention described herein.

Although the foregoing has been described in some detail for purposes of clarity, it will be apparent that certain changes and modifications may be made without departing from the principles thereof. It should be noted that there are many alternative ways of implementing both the processes and apparatuses described herein. Accordingly, the present embodiments are to be considered illustrative and not restrictive.

Those having skill in the art will appreciate that many changes may be made to the details of the above-described embodiments without departing from the underlying principles of the disclosure. The scope of the present disclosure should, therefore, be determined only by the claims, if any.

What is claimed is:

1. A portable endoscope with controlled suction, the endoscope comprising:
a proximal component and a distal component, the proximal component including a handle and the distal component comprising a flexible insertion tube extending from the proximal component to a distal end, the insertion tube including an internal working channel extending from the distal end to the proximal component;
a powered suction system including a fluid circuit in fluid communication with the internal working channel of the proximal component, the powered suction system providing suction to the distal end, the powered suction system including,
a source of negative pressure from a pump;
a liquid trap in the fluid circuit, the liquid trap in fluid communication with the source of negative pressure; and
a suction control system comprising at least one user input element, the suction control system configured to use a sensed pressure to manipulate the negative pressure applied to the working channel.

2. The portable endoscope of claim 1, wherein the powered suction system includes a pressure sensor configured to sense the negative air pressure applied to the trap and the suction control system includes an electrical processing unit electrically coupled to the pressure sensor and providing the sensed pressure.

3. The portable endoscope of claim 1, wherein the suction control system includes a mechanically actuated negative pressure release valve.

4. The portable endoscope as in claim 1, wherein the proximal component includes one or more user input elements that provide input to the suction control system to selectively adjust the regulated negative pressure applied to the working channel of the distal component, wherein the electrical processing unit is responsive to the user input for regulating suction pressure.

5. The portable endoscope as in claim 1, wherein suction control system regulates the pressure applied to the working channel by changing the pump pressure.

6. The portable endoscope as in claim 1, wherein the suction control system regulates the pressure applied to the working channel by releasing pressure in the fluid circuit.

7. The portable endoscope as in claim 1, wherein the pump is located in the proximal component.

8. The portable endoscope as in claim 1, wherein the pump is located in a handle portion of the proximal component.

9. The portable endoscope as in claim 1, wherein the pump is located in an external component in fluid communication with the proximal component.

10. The portable endoscope as in claim 1, wherein the fluid circuit includes an over-pressure valve, wherein the electrical processing unit actuates the over-pressure valve in response to a sensed pressure in the fluid circuit that is greater than a user selected pressure.

11. The portable endoscope as in claim 10, wherein the over-pressure valve is a solenoid valve.

12. The portable endoscope as in claim 1, wherein the suction control system includes the pressure sensor on a circuit board and a manifold mounted to the circuit board over the pressure sensor, the manifold providing fluid communication with an air outlet of the fluid trap and the air inlet to the negative pressure pump.

13. The portable endoscope as in claim 12, wherein the manifold provides fluid communication to an over-pressure valve.

14. The portable endoscope as in claim 1, wherein the proximal end includes a fluid trap port, the port including an inlet connector that is in fluid communication with the working channel and receives liquids and air from the working channel, and an outlet connector that is in fluid communication with the source of negative pressure.

15. The portable endoscope as in claim 1, wherein the inlet connector and the outlet connector are tubing connectors.

16. The portable endoscope as in claim 1, further comprising a hydrophobic filter in the fluid circuit between the outlet connector and the negative pressure pump.

17. The portable endoscope as in claim 1, further comprising an accessory port for inserting fluid in the working channel, the fluid circuit including a fluid valve configured to prevent backflow of liquids from the accessory port toward the source of negative pressure.

18. The portable endoscope of claim 1, further comprising a flexion control system including a thumb lever, a flexion wheel, and a plurality of steering wires, the flexion control system providing user control of the flexible tubing of the distal component.

19. A portable bronchoscope with controlled suction of the airway, the bronchoscope comprising:
a proximal component and a distal component, the proximal component including a handle and the distal component comprising a flexible insertion tube extending from the proximal component to a distal end, the insertion tube including an internal working channel extending from the distal end to the proximal component;
a powered suction system including a fluid circuit in fluid communication with the internal working channel of the proximal component, the powered suction system providing suction to the distal end, the powered suction system including,
a negative pressure pump positioned in a handle portion of the proximal component;
a liquid trap in the fluid circuit, the liquid trap in fluid communication with the source of negative pressure;
a pressure sensor configured to sense the negative air pressure applied to the liquid trap;
a suction control system including an electrical processing unit electrically coupled to the pressure sensor and responsive to user input for regulating suction pressure, the suction control system configured to use the sensed pressure to regulate the negative pressure applied to the working channel;
wherein the proximal component includes,
a flexion control system including a thumb lever, a flexion wheel, and a plurality of steering wires, the flexion control system providing user control of the flexible tubing of the distal component; and
one or more user input elements that provide input to the suction control system to selectively adjust the regulated negative pressure applied to the working channel of the distal component; and
wherein the suction control system regulates the pressure applied to the working channel by (i) changing the negative pressure generated by the pump and/or (ii) releasing pressure in the fluid circuit.

20. A portable endoscope, comprising:
a proximal component and a distal component, the proximal component including a handle and the distal component comprising a flexible insertion tube extending from the proximal component to a distal end, the insertion tube including an internal working channel extending from the distal end to the proximal component;
a powered suction system including a fluid circuit in fluid communication with the internal working channel of the proximal component, the powered suction system providing suction to the distal end, the powered suction system including,
a negative pressure pump positioned in the proximal component;
a liquid trap in the fluid circuit, the liquid trap in fluid communication with the pump; and
a suction control system comprising at least one user input element, the suction control system configured modulate the negative pressure applied to the working channel.
